(19) United States
(12) Patent Application Publication
(10) Pub. No.: US 2013/0131462 A1
(43) Pub. Date: May 23, 2013

(54) DEVICE FOR MODULAR ANALYSIS

(71) Applicants: Kristin Klückmann, Lubeck (DE); Martin Hofler, Hamburg (DE); Manfred Müller, Kiel (DE); Anja Bosy-Westphal, Kiel (DE)

(72) Inventors: Kristin Klückmann, Lubeck (DE); Martin Hofler, Hamburg (DE); Manfred Müller, Kiel (DE); Anja Bosy-Westphal, Kiel (DE)

(73) Assignee: SECA AG, Reinach BL 1 (CH)

(21) Appl. No.: 13/700,952

(22) PCT Filed: May 30, 2011
(86) PCT No.: PCT/DE2011/001137

§ 371 Date: Feb. 6, 2013

(30) Foreign Application Priority Data
May 31, 2010 (DE) 10 2010 022 637.8

(51) Int. Cl. A61B 5/0205; A61B 5/01; A61B 5/0402; A61B 5/1455; A61B 5/053; A61B 5/021

(52) U.S. Cl. CPC A61B 5/02055; A61B 5/053; A61B 5/021; A61B 5/0402; A61B 5/14551; A61B 5/01
USPC 600/301; 600/547; 600/485; 600/509; 600/323; 600/549

(57) ABSTRACT

The device is provided with at least one analysis apparatus for analyzing at least one biological parameter of a living being. The analysis apparatus has at least one data input for recording measured data of at least one sensor. The sensor measures at least one biological parameter of the living being. The analysis apparatus also has at least one output apparatus for an analysis result. The analysis apparatus has a control unit and a program memory. The program memory stores a plurality of program modules, which can be activated by the control unit according to an externally specifiable control instruction alternatively or in partial or complete combination with each other in such a way that the activated program modules provide the data for the selected analysis result.

The invention pertains to a device with at least one evaluation apparatus for evaluating at least one biological parameter of a living organism, which device comprises at least one data input for acquiring measurement data from at least one sensor, which measures at least one biological parameter of the living organism, and which device also comprises at least one output apparatus for an evaluation result.

Devices of this type are especially suitable for providing measurement values pertaining to the health or nutritional status of a patient. Known devices comprise for this purpose an unchangeable structure adapted to the application in question and are often provided with appropriate general control software.

The goal of the present invention is to design a device of the type described above in such a way that evaluation results which pertain to specifiable questions can be provided.

This goal is achieved according to the invention in that the evaluation apparatus comprises a control unit and a program memory, wherein the program memory contains a plurality of program modules, some or all of which can be activated as desired in combination with each other by the control unit as a function of an externally specifiable control instruction in such a way that the activated program modules provide the data for the selected evaluation result.

The inventive device thus comprises a modular structure, such that the hardware or software modules required in a particular case can be activated and linked together. The device can, for example, have the basic structure of an apparatus for analyzing body composition (“body composition analyzer”). Other possible applications include, for example, scales and height-measuring devices.

A modular system design is facilitated by connecting the sensor and the evaluation apparatus to each other by a data transfer link.

Another way of enhancing the modular design of the system is to connect a display apparatus and the evaluation apparatus to each other by a data transfer link.

The processing of the existing data is facilitated by providing the evaluation apparatus with an interface for the input of data not acquired metrologically.

It is especially suitable for the interface to be designed for manual data entry.

The system can be provided with comprehensive configurability by selecting the at least one sensor so that it can metrologically determine at least one measurement parameter selected from the group: body weight, impedance, height, blood pressure, ECG, heart rate, blood values, pulse oximetry, temperature, respiratory parameters, auscultatory parameters, and/or energy consumption.

Evaluation results can be provided immediately by connecting the evaluation unit to a printer.

Exemplary embodiments of the invention are illustrated schematically in the drawings:

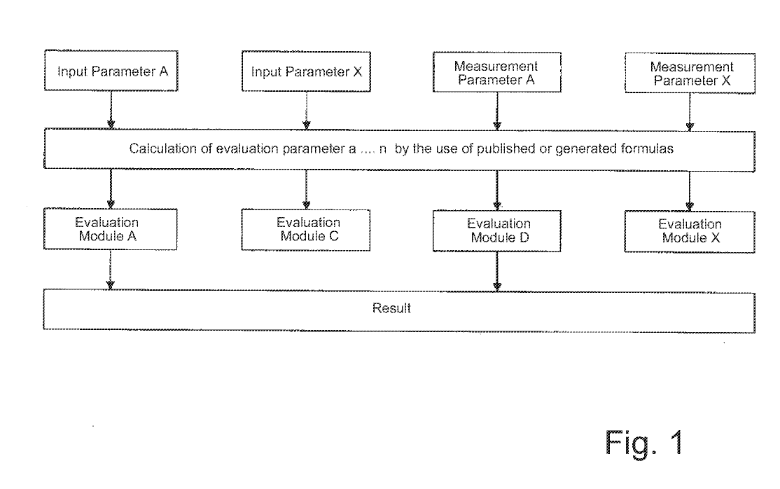

FIG. 1 shows a block circuit diagram illustrating the basic design of the device;

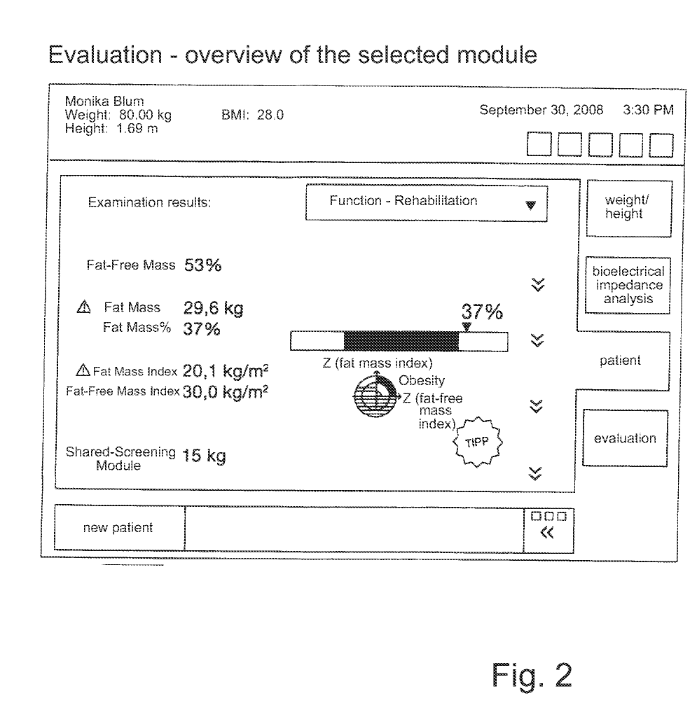

FIG. 2 shows an example of how the evaluation results are displayed;

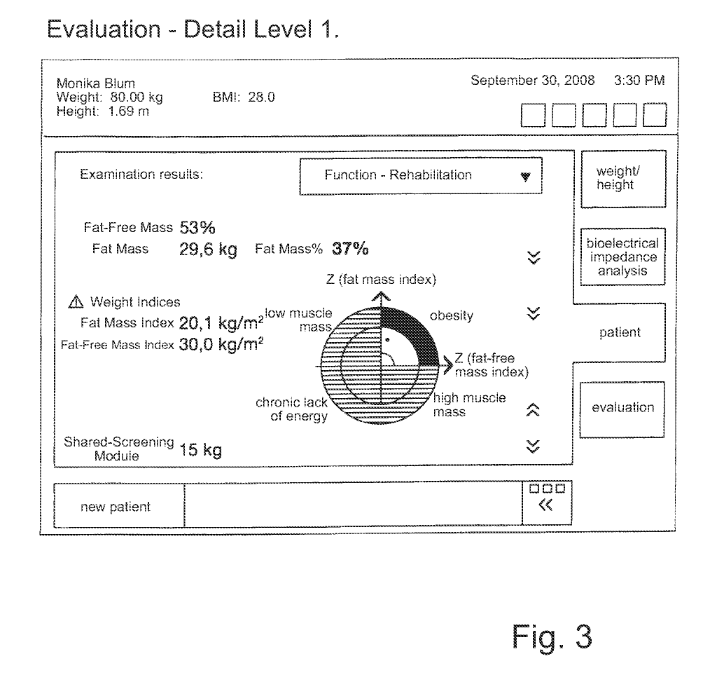

FIG. 3 shows an example of how detailed evaluation results are displayed on a first level of detail;

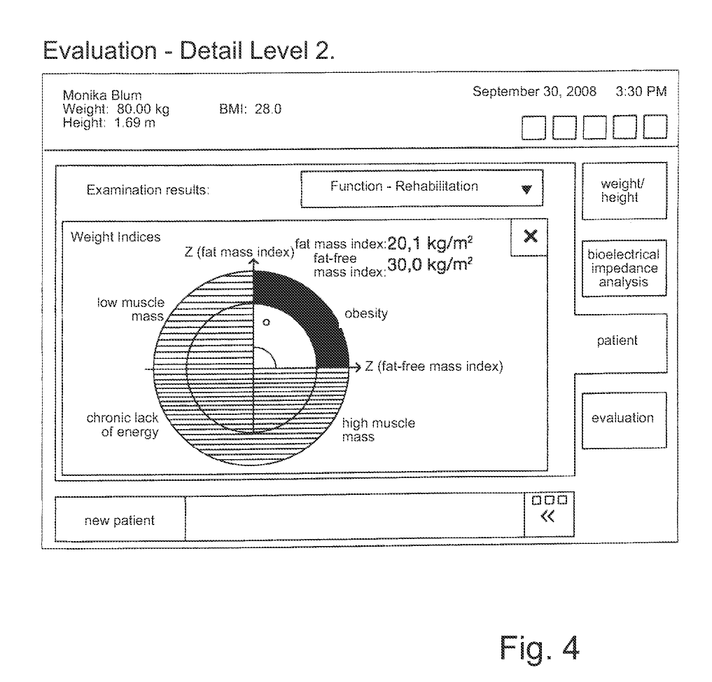

FIG. 4 shows an example of how detailed evaluation results are displayed on a second level of detail;

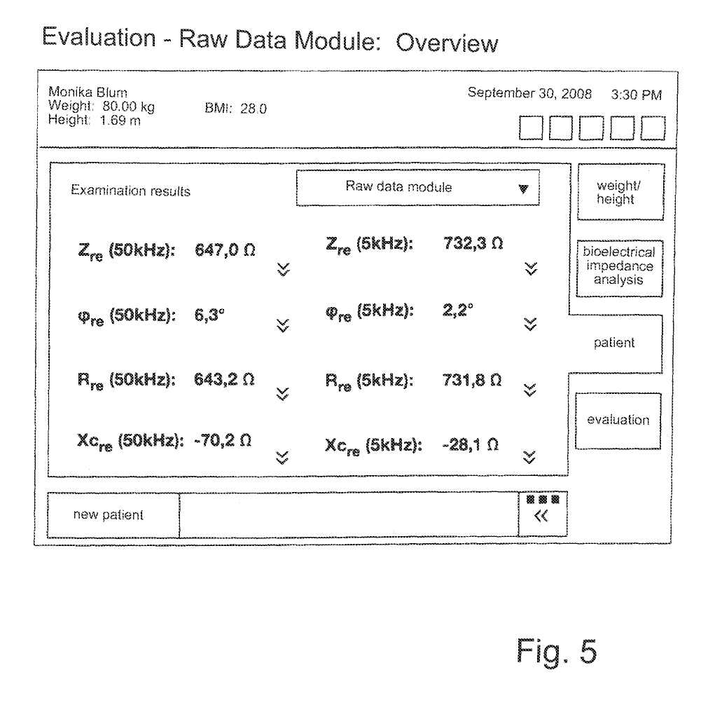

FIG. 5 shows an example of how the raw data of the evaluation are displayed;

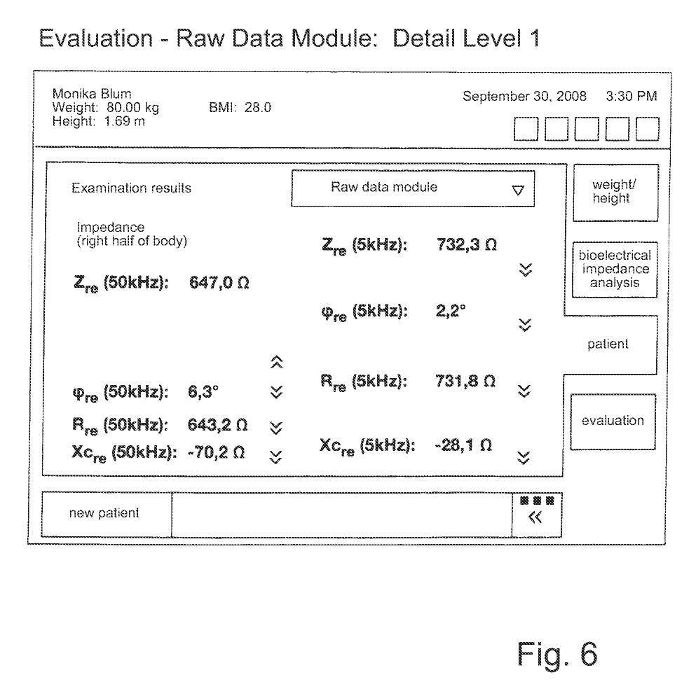

FIG. 6 shows a detailed diagram of the raw data of the evaluation;

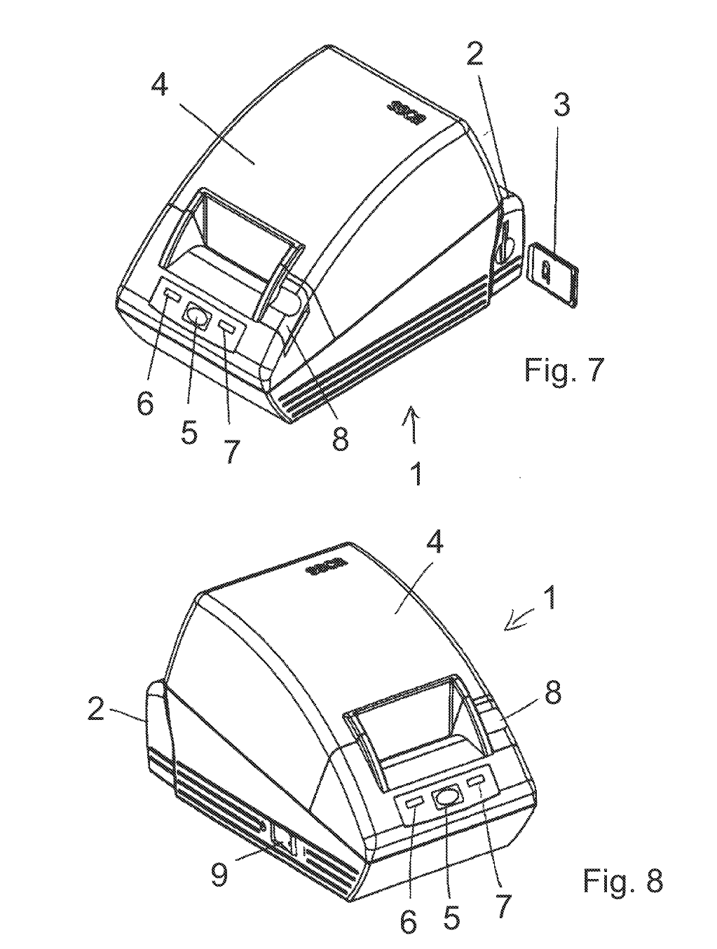

FIG. 7 shows a perspective view of a printer for data output with an adapter and a data card; and

FIG. 8 shows another perspective view of a printer according to FIG. 7.

The inventive device has at least one measuring instrument, i.e., at least one sensor, for at least one of the following parameters of a patient: weight, impedance, height, blood pressure, ECG, heart rate, blood values, pulse oximetry, temperature, respiratory parameters, auscultatory parameters, and/or energy consumption. In general, the measurement parameters can pertain to any desired physical variables of the patient determined within the scope of a medical examination. The measurement parameters can be sent directly to the evaluation apparatus or sent by way of external data sources such as a laboratory data system.

In addition to the measurement parameters, input parameters can also be used, which are determined on the basis of a patient odentification step or by the answers to questions asked before the examination. These can be questions about, for example, the sex and/or the age and/or the ethnicity of the person.

Evaluation parameters are determined on the basis of the measurement parameters and/or the input parameters. This can be done, for example, by the use of mathematical formulas implemented in an evaluation apparatus, which determine the associated values for the evaluation parameters. The formulas can be taken from the published prior art, for example, or they can be determined on the basis of clinical trials. The evaluation parameters are interpreted by the use of references. These references are normal ranges, which are published in scientific articles or determined by series of measurements. An example of a graphic representation of this type relating to a reference value is the phase angle.

By the use of evaluation modules, it is possible to combine different evaluation parameters with each other. The evaluation modules serve to provide the values for the specific questions to be answered. One such question can pertain to the energy status of a person.

As a function of the selection made by the operator of the device, the evaluation modules relevant to the examination situation in the actual case at hand are combined with each other.

FIG. 1 illustrates the appropriate linking of the input parameters with the measurement parameters via the calculation of the evaluation parameters and the corresponding provision of the evaluation modules for deriving the result.

The hardware components being used can be connected by, for example, a wireless network, especially with the use of USB wireless adapters. Switching between different wireless networks is possible. It is also possible to connect several workplaces together, preferably by an ethernet network.

FIG. 2 shows a display screen of the device for a female patient as an example. With respect to the evaluation, an overall view of the selected module is illustrated.

FIG. 3 shows the display screen of a first level of detail of an evaluation serving as an example. Being able to visualize the results makes it easier to see whether selected parameters are within a tolerance range or not.

FIG. 4 shows a second level of detail for the evaluation with a magnified visualized graphic evaluation.

FIG. 5 shows an overall view of the raw data module of the evaluation.

FIG. 6 shows, for further illustration, detail level 1 of the raw data evaluation module.

In the inventive device, the selected sensor or the measuring instrument being used is connected to the assigned evaluation unit by a data transfer link. The data transfer link can be realized in wireless or wired fashion. Both the evaluation unit and the display unit have a functionally modular design, so that whatever functionality is needed can be easily configured.

The evaluation apparatus can be realized by, for example, an appropriately programmed computer. In particular, it is also possible to implement the evaluation unit in the area of an evaluation component, which can be connected to a printer. This makes it possible to reach an immediate decision about the evaluated parameters.

With respect to the display of the determined values, it is especially effective for the data which suggest certain health risks to be emphasized. This can be done by displaying them in a larger size, by using a different color, or by varying the type of display over time.

FIG. 7 shows a printer 1, which is designed to serve here as part of the evaluation apparatus or which provides the evaluation unit or which can be connected to the evaluation apparatus. According to the illustrated embodiment, the printer 1 is connected to an adapter 2, which provides the evaluation unit. In particular, it is advisable for the adapter 2 to enable wireless communication with one or more measuring instruments.

According to another preferred embodiment, the adapter 2 is plugged into a standard interface of the printer. This can be a parallel interface, for example, a serial interface, or a USB port.

According to another preferred embodiment, the adapter 2 comprises a universal hardware structure and is configured by an additional element 3 as a function of the application. It is advisable in particular for the additional element 3 to be provided with control and/or evaluation software. For example, the additional element 3 can be in the form of an SD card.

According to a typical embodiment of the printer 1, it has a door 4 to allow the insertion of a roll of paper. An operating element 5 and display elements 6, 7 are also provided. A button 8 is used to unlock the door 4.

FIG. 8 shows a different perspective view of the printer. Here a switch 9 for turning the machine on and off can also be seen.

According to one embodiment of the evaluation apparatus, it is provided that the unit configures itself as a function of the acquired metrological parameters. This self-configuration can be completely automatic, or the configuration can be suggested to the operator. During a preliminary examination of the measurement values, it is determined which modules are suitable for the concrete case at hand as a function of the metrologically detected situation.

According to another variant, it is provided that, to minimize the required evaluation time as a function of the modules selected during the configuration step, only the measurement values required for the modules which have in fact been activated are determined and evaluated.

According to an exemplary embodiment, the use of the adapter 2 makes it possible for the printer 1 to calculate automatically the parameters of the program module for which the required input values and measurement values are available. It is also possible to use the adapter 2 to define which modules are to appear on the printout of the printer 1 by means of a module menu previously configured in the machine.

A program module is typically defined by a predetermined number of parameters. A parameter is in this case a measurement value or a calculated value, which yields information on the health status of a person. Different parameters can be assembled freely into a user-specific module by using a menu.

1-10. (canceled)

11. A device comprising at least one evaluation apparatus for evaluating at least one biological parameter of a living organism, which apparatus includes at least one data input for acquiring measurement data from at least one sensor, which measures at least one biological parameter of the living organism, and at least one output apparatus for an evaluation result, wherein the evaluation apparatus further includes a control unit and a program memory, wherein the program memory contains a plurality of program modules that are activateable selectively or in partial or complete combination by the control unit as a function of an externally specifiable control instruction so that the activated program modules provide data for the selected evaluation result.

12. The device according to claim 11, wherein the sensor and the evaluation feature are connected to each other by a data transfer link.

13. The device according to claim 11, wherein a display apparatus and the evaluation apparatus are connected to each other by a data transfer link.

14. The device according to claim 11, wherein the evaluation apparatus comprises an interface for an input of data not metrologically acquired.

15. The device according to claim 14, wherein the interface is configured for manual data entry.

16. The device according to claim 11, wherein the at least one sensor metrologically acquires at least one measurement parameter selected from the group consisting of: body weight; impedance; height; blood pressure; ECG; heart rate; blood composition; pulse oximetry; temperature; respiratory parameters; auscultatory parameters; and/or energy consumption.

17. The device according to claim 11, further comprising a printer connected to the evaluation apparatus.

18. The device according to claim 17, wherein the evaluation apparatus is located in an area of an adapter of the printer.

19. The device according to claim 18, wherein the adapter is configurable by way of an additional element.

20. The device according to claim 11, wherein the evaluation apparatus is operative to activate and deactivate sensors and/or evaluation modules for sensors.
